(19) United States
(12) Patent Application Publication
(10) Pub. No.: US 2025/0388979 A1
(43) Pub. Date: Dec. 25, 2025

(54) METHOD FOR RAPIDLY TESTING BIOLOGICAL ACTIVITY OF HUMAN INTERLEUKIN-11 ON BASIS OF LUCIFERASE REPORTER GENES, AND CONSTRUCTION METHOD AND APPLICATION OF CELL STRAINS

(71) Applicant: CHINA JILIANG UNIVERSITY, Zhejiang (CN)

(72) Inventors: Yafen Zhang, Zhejiang (CN); Zihong Ye, Zhejiang (CN); Yiying Wang, Zhejiang (CN); Jintian Tang, Zhejiang (CN)

(73) Assignee: CHINA JILIANG UNIVERSITY, Zhejiang (CN)

(21) Appl. No.: 19/235,522

(22) Filed: Jun. 11, 2025

(30) Foreign Application Priority Data
Jun. 19, 2024 (CN) 202410791811.1

(51) Int. Cl. C12Q 1/6897; C12N 15/70; C12N 15/85

(52) U.S. Cl. CPC C12Q 1/6897; C12N 15/70; C12N 15/85

(57) ABSTRACT

The present disclosure provides a method for rapidly testing biological activity of human interleukin-11 on the basis of luciferase reporter genes, and a construction method and application of cell strains. The method includes the following steps: 1) constructing cell strains based on luciferase reporter genes; and 2) preparing a sample to be tested into a gradient dilution solution, adding same into a culture solution, then, adding the culture solution into a culture medium containing the cell strains, and after co-incubation, testing the biological activity of human interleukin-11 by determining luciferase activity. The present disclosure successfully constructs a new method for testing the biological activity of interleukin-11 (IL-11). Experimental time is shortened from 48-56 h to 24-28 h by the constructed RGA method, and working efficiency is obviously improved, such that the time cost is saved.

### CROSS-REFERENCE TO RELATED APPLICATION

This application claims the priority benefit of China application no. 202410791811.1 filed on Jun. 19, 2024. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Oct. 7, 2024, is named 149607_SEQUENCELISTING and is 4,721 bytes in size.

### BACKGROUND OF THE INVENTION

### Technical Field

The present disclosure belongs to the field of biotechnology, and particularly relates to a method for testing biological activity of human interleukin-11 on the basis of luciferase reporter genes, and a construction method and application of cell strains.

### Background of Related Art

In recent years, protein biologics have developed rapidly, showing a continuous growth trend in the field of medicine. According to the latest scientific research and market analysis, protein biologics have become an important means of treating a variety of diseases which cover cancer, autoimmune diseases, infectious diseases and other fields. Driven by biotechnology, more and more new technologies and innovations continue to emerge, providing more possibilities for development and production of the protein biologics.

Interleukin-11 (IL-11) is one of more than 30 interleukins discovered, belongs to the IL-6 family and can be used as an anti-inflammatory agent against various inflammatory diseases. Clinical studies have found that IL-11 is closely related to the occurrence, development, metastasis and tumor cell differentiation of some cancers, such as colon cancer, esophageal squamous cell carcinoma (ESCC), and breast cancer. IL-11 has been confirmed as an important factor and a potential molecular mechanism in these cancers. IL-11 is not only closely related to the pathogenesis of solid malignant tumors, but also can be used as an anti-inflammatory agent against various inflammatory diseases. For example, IL-11 plays a key role in autoimmune inflammatory diseases, including rheumatoid arthritis, multiple sclerosis, diabetes, and systemic sclerosis, as well as other chronic inflammatory diseases, such as periodontitis, asthma, chronic obstructive pulmonary disease, psoriasis, and colitis.

At present, a method for testing biological activity of IL-11 in Chinese Pharmacopoeia is a cell proliferation inhibition method, which tests the biological activity of human interleukin-11 according to different proliferation speeds of mouse B9 hybridomasubcloning cell strains (B9-11 cell strains) under different concentrations of human interleukin-11 (IL-11). But rhIL-6 can also proliferate B9-11 cells. Since IL-11 and IL-6 have some common biological activity, in order to improve specificity of IL-11 testing, anti-IL-6 monoclonal antibody should be used for neutralizing IL-6 activity in each sample test to ensure the accuracy and specificity of experimental results, which not only requires use of a toxic MTT reagent, but also requires testing time to be 48-56 h. Therefore, it is necessary to establish a more rapid and accurate method to test the biological activity of IL-11.

### SUMMARY OF THE INVENTION

Aiming at the problems existing in the prior art, an objective of the present disclosure is to provide a method for testing biological activity of human interleukin-11 on the basis of luciferase reporter genes, and a construction method and application of cell strains, which are specifically implemented through the following technical solutions:

A method for rapidly testing biological activity of human interleukin-11 on the basis of luciferase reporter genes is provided and includes the following steps:
1) constructing cell strains based on luciferase reporter genes; and
2) preparing a sample to be tested into a gradient dilution solution, adding same into a culture solution, then, adding the culture solution into a culture medium containing the cell strains, and after co-incubation, testing the biological activity of human interleukin-11 by determining luciferase activity.

A construction method for cell strains based on luciferase reporter genes includes the following steps:
1) constructing pPR(EXP)-STAT3-luc2 plasmids, which includes the specific steps:
1-1) synthesizing a promoter: obtaining STAT3 gene action element fragments;
1-2) performing connection and transformation: firstly, digesting a pPR(EXP)-luc2 vector with EcoR I and Sal I, then ligating the digested pPR(EXP)-luc2 vector with the STAT3 gene promoter fragment, and performing transformation into Escherichia coli competent cells after completion of ligation;
1-3) performing screening and verification: spreading the transformed Escherichia coli on a plate containing an antibiotic, performing inverted culture for 12-16 h, selecting bacteria for PCR identification, extracting plasmids from positive colonies, performing digestion to identify recombinant plasmids, sequence positive clones, and extract plasmids after sequencing and alignment is successful, thereby obtaining constructed recombinant expression plasmids of pPR(EXP)-STAT3-luc2;
2) transfecting cells with the plasmids of pPR (EXP)-STAT3-luc2, which includes the specific steps:
2-1) performing recovery and culture of HEK293T cells: dissolving cryopreserved HEK293T cells in a 37° C. water bath, performing centrifugation for 5 min at 1360 rpm after the cells are dissolved, removing supernatant through suction, adding 1 mL of a complete medium of cells for resuspension, performing transfer into a T25 culture flask containing 5 mL of a complete medium of cells, and performing culture in an incubator;
2-2) performing cell transfection: seeding the HEK293T cells, performing culture for 24 h, then, adding the cells into a transfection system for culture for 48 h at 37° C., discarding a transfection solution, adding a culture solution, performing passaging at a density of 1:4 when the cells grow close to confluence, continuing culture, and performing positive monoclonal screening when cell confluency reaches 30% to obtain transfected cells;
3) screening stable cell lines, which includes the specific steps:
3-1) screening puromycin: washing the transfected cells with phosphate buffer saline (PBS), performing digestion with trypsin, inoculating the cells into a culture dish, then, adding a culture medium containing puromycin, replacing the selective medium every 2-3 days, transferring the cells to a culture flask for continuous culture when the cell confluency is more than 90%, adding a culture medium containing puromycin, selecting monoclonal cells for continuous culture when the transfected cells grow to more than 70%, and adding a culture medium containing puromycin, where a concentration of the puromycin is reduced by half at this time; and
3-2) performing monoclonal selection and expanded culture: screening positive clones by a limited dilution method to obtain the cell strains.

Further, in step 1-1), by consulting literature and NCBI, JASPAR is utilized to predict a promoter region, STAT3 related action elements are screened, four segments of sequences most closely related to human interleukin-11 action are selected, and cleavage sites of EcoR I and Sal I are added at both ends of the sequences respectively after the sequences are connected in series as action elements.

Further, in step 1-2), DH5α competent cells are used as the Escherichia coli competent cells.

Further, in step 1-3), the antibiotic is ampicillin.

Further, in step 2-1), the complete medium of cells is a Dulbecco's modified eagle medium (DMEM) culture solution containing double antibiotics of 10% fetal bovine serum and 1% streptomycin, and the culture conditions in the incubator are 5% CO₂ and 37° C.

Further, in step 2-2), the transfection system includes 2.5 μg of the pPR (EXP)-STAT3-luc2 plasmid, 1.25 μgof a transposon enzyme plasmids, 5 μL of a P3000 reagent, 3.75 μL of a Lipofectamine 3000 reagent and 250 μL of a DMEM.

Further, in step 3-2), the steps of screening positive clones by the limiting dilution method include: preparing cell suspension, performing cell counting, diluting the cells to 5 cells/mL with a puro culture solution, adding the cell suspension into a 96-well plate at 200 μL/well, where a puro concentration is 25 μg/mL, then, adding the cell suspension at 100 μL/well, labeling monoclonal clones under a microscope, after 96 wells are full of cells, transferring the cells into new wells for culture, performing repeated screening, and when the cultured cells basically do not die, performing sequencing identification to obtain the cell strains.

Application of the above-mentioned cell strains based on luciferase reporter genes in testing of biological activity of human interleukin-11 (IL-11) is provided, which is high in specificity and sensitivity, and is expected to become common biological activity testing of IL-11.

The present disclosure successfully constructs a new method for testing the biological activity of IL-11. Compared with a traditional cell proliferation inhibition method, the constructed RGA method is used as a means for rapidly testing the activity of rhIL-11, experimental time is shortened from 48-56 h to 24-28 h by the RGA method, and working efficiency is obviously improved, such that precious time cost is saved. In addition, the method provided by the present disclosure completely avoids use of toxic reagents, thereby ensuring safety of experimental personnel. The method provided by the present disclosure has advantages over the conventional cell proliferation inhibition method in simplicity, sensitivity and cost.

### BRIEF DESCRIPTION OF THE DRAWINGS

FIG. 1 is a map of pPR (EXP)-STAT3 reporter gene plasmids constructed in Example 2.

FIG. 2 shows digestion identification of pPR(EXP)-STAT3 recombinant plasmids in Example 2 (in the figure, A shows double digestion verification, and B is undigested pPR (EXP)-STAT3 plasmids).

FIG. 3 shows response of HEK293T cells transiently transfected with pPR (EXP)-STAT3 to rhIL-11.

FIG. 4 shows response of HEK293T cells stably transfected with pPR(EXP)-STAT3-luc2-puro to rhIL-11.

FIG. 5 shows isolation and screening of monoclonal cell strains (A shows chemiluminescence intensity of the monoclonal cell strains, and B shows a reaction curve of the monoclonal cell strains to rhIL-11).

FIG. 6 shows cell signaling pathway verification by JAK-STAT3 inhibitor.

FIG. 7 shows optimization of a pre-dilution factor and a dilution gradient to optimize the inoculation quantity of cells (A shows rhIL-11 reaction curves under different dilution gradients, and B shows signal-to-noise ratios under different dilution gradients).

FIG. 8 shows optimization of an inoculation quantity of cells (A shows reaction curves to rhIL-11 under different cell numbers, and B shows signal-to-noise ratios of response to rhIL-11 under different cell numbers).

FIG. 9 shows optimization of rhIL-11 action time (A shows reaction curves under different incubation time, and B shows signal-to-noise ratios under different incubation time).

FIG. 10 shows within-day precision of Sample 1.

FIG. 11 shows within-day precision of sample 2.

FIG. 12 shows inter-day precision reactivity comparison of sample 1.

FIG. 13 shows inter-day precision reactivity comparison of sample 2.

FIG. 14 shows cell specificity for denatured rhIL-11.

FIG. 15 shows cell specificity for antibodies to different targets.

FIG. 16 shows stability of 293T-G11 cells in a selective medium (A shows luciferase expression amounts of cells of different generations stimulated by sample 1 in the selective medium, B shows luciferase expression amounts of cells of different generations stimulated by sample 2 in the selective medium, and C shows biological activity of samples tested by cells of different generations in the selective medium).

FIG. 17 shows comparison of reactivity between cells of different generations in a

selective medium (A shows the reactivity of sample 1 in the selective medium, and B shows the reactivity of sample 2 in the selective medium).

FIG. 18 shows stability of 293T-G11 cells in a growth medium (A shows luciferase expression amounts of cells of different generations stimulated by sample 1 in the growth medium, B shows luciferase expression amounts of cells of different generations stimulated by sample 2 in the growth medium, and C shows biological activity of samples tested by cells of different generations in the growth medium).

FIG. 19 shows robustness comparison of reactivity of cells of different generations in a growth medium (A shows the reactivity of sample 1 in the growth medium, and B shows the reactivity of sample 2 in the growth medium).

FIG. 20 shows robustness evaluation (A shows luciferase expression under different incubation time, B shows signal-to-noise ratios under different incubation time, C shows luciferase expression under different luciferase substrate amounts, D shows signal-to-noise ratios under different luciferase substrate amounts, E shows luciferase expression at different testing action time of luciferase substrates, and F shows signal-to-noise ratios at different testing action time of luciferase substrates).

FIG. 21 shows suitability evaluation (A shows reaction curves under different substrates, and B shows signal-to-noise ratios under different substrates).

FIG. 22 shows comparison between RGA and a proliferation inhibition method.

### DETAILED DESCRIPTION OF THE EMBODIMENTS

The present disclosure is further described below with reference to particular examples, so as to understand the technical solution better.

### Example 1: Construction of Reporter Gene Cell Strains

1. Construction of reporter gene plasmids: by consulting literature and NCBI, JASPAR was utilized to predict a promoter region, STAT3 related action elements were screened, four segments of sequences most closely related to human interleukin-11 action were selected, and cleavage sites of EcoR I and Sal I were added at both ends of the sequences respectively after the sequences were connected in series. Synthesis was made by Suzhou Haichen Biotechnology Company.

IL-11Rα was retrieved at NCBI, CDS sequences of IL-11Rα genes were inserted into common pPR plasmids, and synthesis was made by Suzhou Haichen Biotechnology Company.

STAT3 action elements and the plasmids containing IL-11Rα genes were digested by restriction endonuclease and connected by T4DNA ligase to obtain the reporter gene plasmids required by the present disclosure.

2. Cell transfection: the constructed plasmids were mixed with transposon enzyme plasmids at 2:1, and then, transfection was performed according to the following steps:
1) Seeding: cells were seeded into a six-well plate with 5×10⁵ cells for each well, and supplementation was made to 2 mL with a complete Dulbecco's modified eagle medium (DMEM) for culture at 37° C., and 5% CO₂ until the density reached 70%-80%.
2) Starvation treatment: the cell growth medium in the six-well plate was removed through suction and replaced with a basal DMEM without foetal bovine serum (FBS) and double antibiotics, and culture was performed at 37° C. and 5% CO₂ for 6-8 h.
3) Lipo3000 was mixed with the plasmids according to the proportion of the description, mixed evenly, and kept standing for 15-20 min.
4) The culture solution in the six-well plate was removed through suction, and rinsing was performed twice with phosphate buffer saline (PBS). 1.5 mL of basal DMEM without serum and double antibiotics, and the lipo3000 plasmid mixed solution prepared according to the description were added into each well for culture at 37° C. and 5% CO₂ for 48 h.
5) Liquid in the six-well plate was removed through suction, and replaced with a complete DMEM.

### 3. Monoclonal Cell Strain Screening

### 1) Determination of Puromycin Screening Concentration

1-1) 293T cells in a logarithmic growth phase were taken, the concentration was adjusted to 20000 cells per ml after cell counting, and the cells were added into a 24-well plate with 1 mL per well.
1-2) After culture for 24 h, the culture medium of each well was replaced with 1 mL of a

complete medium. A puromycin mother solution was added into each well, a concentration range was 0.5 μg/μL-10 μg/μL, and 3 duplicate wells in parallel was made.
1-3) Operation in 1-2) was repeated every 3 d.
1-4) After 2 weeks, survival situations of the cells were observed to determine the screening concentration of puromycin.

### 2) Pressure Screening

2-1) 48 h after transfection, a pipette was utilized to remove the complete medium, and washing once with PBS. A selective medium was added at 2 mL/well, and the medium was replaced once every 3 d.
2-2) After the cells in the 6-well plate were fully grown, all the cells were transferred to a T25 culture flask for culture. After the cells in the T25 culture flask were fully grown, all the cells were transferred to a T75 culture flask for culture. After the cells were fully grown, passaging was carried out.
2-3) Passaging: passaging was performed according to an ordinary 293T cell passaging method, and culture was performed at the conditions of 37° C. and 5% CO₂.

### 3) Monoclonal Cell Isolation

3-1) After the screened mixed clonal cells could be stably passaged, cells were collected, and supernatant was discarded.
3-2) A selective medium was added to resuspend the cells, and the cell density was reduced to 5 cells/mL.
3-3) The diluted cells were inoculated a 96-well plate at 200 μL per well.
3-4) Culture was performed at 37° C. and 5% CO₂ for 2 h, then, observation was performed under a microscope, and wells with single cells were marked.
3-5) Cultured was performed for 15-20 d, and when the monoclonal cells in the 96-well plate were fully grown, all the cells in the wells were transferred to a 24-well plate. Then, a selective medium was added at 1 mL/well, and culture was performed at 37° C. and 5% CO₂.
3-6) When the cells in the 24-well plate were fully grown, all the cells were transferred to a 6-well plate for culture. When the cells in the 6-well plate were fully grown, all the cells were transferred to a T25 culture flask for culture. After the cells in the T25 culture flask were fully grown, all the cells were transferred to a T25 culture flask for culture, and a new T75 culture flask was taken for culture and passaging. 2 successive generations were passaged.
3-7) Luciferase assay was made to test the reactivity of monoclonal cell strains to rhIL-11, and the cell strains with good reaction were frozen and stored in a liquid nitrogen tank. ps 4) HEK293T Cell Luciferase Assay
4-1) 293T-G11 cells were cultured in a T75 culture flask, and the cells were collected when the cell confluency reached 70-80%.
4-2) Cell inoculation: the cells were inoculated into a 96-well plate with 4×10⁴ cells per well, and culture was performed at 37° C. and 5% CO₂ for 17-24 h.
4-3) rhIL-11 samples were dissolved in water for injection, a concentration of the samples was adjusted to 1000 IU/mL with a complete medium, and 4-fold serial dilution was performed with 14 gradients in total. The diluted rhIL-11 sample was added to the 96-well plate of step 2) with 100 μL per well, 3 duplicate wells in parallel was made, and incubation was performed at 37° C. and 5% CO₂ for 5-6 h.
4-4) Luminescence intensity was measured by a multifunctional microplate reader according to the operation of the luciferase assay kit.

### Example 2: Construction of Reporter Gene Cell Strains

1. Plasmid construction: SIE, GAS, SBE, and STAT3element were selected as STAT3 action elements in this study by means of literature review and promoter region prediction. Sequences were as follows:

SIE:
(SEQ ID NO: 1)
5′-GTCGCATTCCCGTCAATC-3′
GAS:
(SEQ ID NO: 2)
5′-GCTGTATTTCCTGAAAGAAGTT-3′
SBE:
(SEQ ID NO: 3)
5′-ACGCGTTACGGGAATCG-3′
STAT3 element:
(SEQ ID NO: 4)
5′-GGCAAGTTCCAGGAATTAAGC-3′

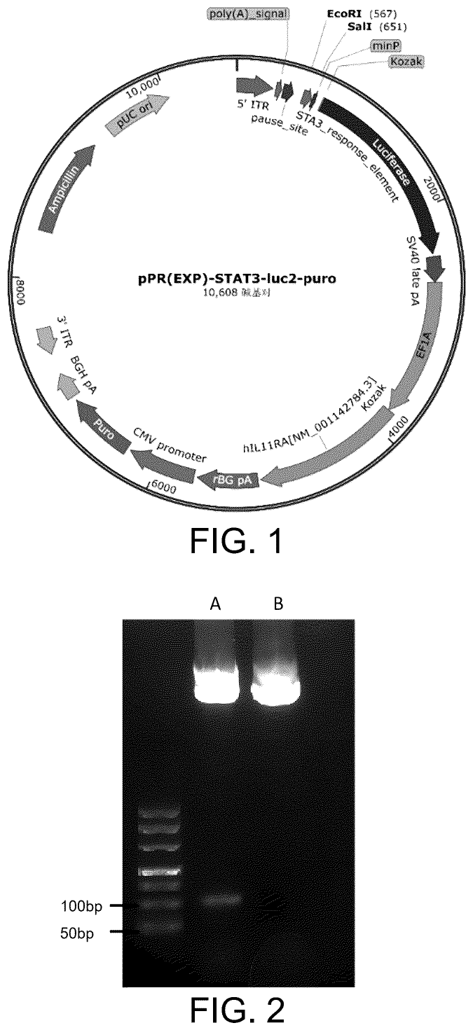

The above sequences were connected in series, restriction enzymes (EcoR I, Sal I) cleavage sites were added at both ends respectively, and the above action elements were ligated into expression vectors with IL-11Rα receptor, Luciferase and Puromycin resistance genes through enzyme digestion and ligation. The plasmids were named pPR(EXP)-STAT3 plasmids, and the plasmid map was shown in FIG. 1.

After restriction enzyme digestion of the constructed pPR(EXP)-STAT3 plasmids, results are shown in FIG. 2. A length of the STAT3 action element fragment is 78 bp, the leftmost is DL500 DNA marker, the middle is a double enzyme digestion band, and the rightmost is an undigested band. The experiment proves that the DNA sequence linked to the multiple cloning sites of the pPR (EXP)-STAT3 vector is consistent with the length of the action element, and is consistent with the theoretical sequence through sequencing verification of Youkang Company.

### 2. Cell Transfection and Polyclonal Cell Testing

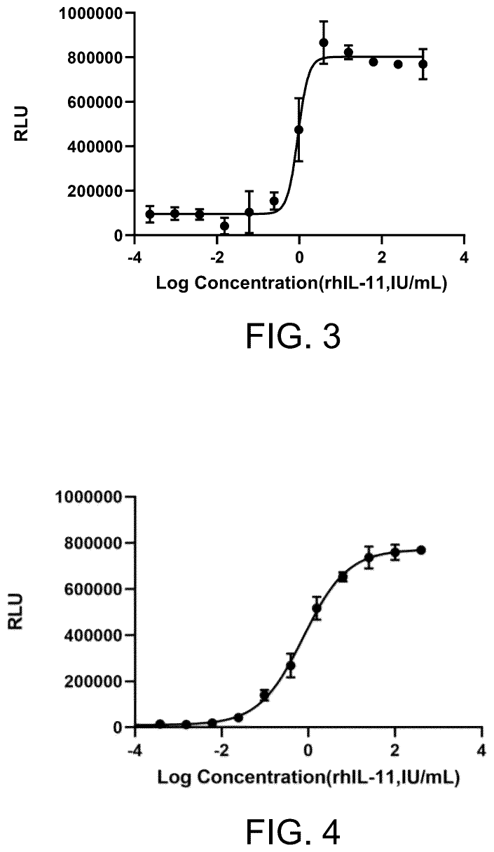

2-1) Transient transfection cell testing: 293T cells transfected with pPR(EXP)-STAT3 plasmids were stimulated by rhIL-11. There was a dose-response relationship between a chemiluminescence intensity and rhIL-11 concentration, and a four-parameter curve could be fitted according to the chemiluminescence intensity. Results are shown in FIG. 3. The chemiluminescence intensity reaches the maximum under the stimulation of rhIL-11 at 12.35 IU/mL, EC₅₀ is 0.9293, R² is 0.966, and the experiment is relatively reliable. The above experimental results demonstrate that the 293T cells transfected with the pPR(EXP)-STAT3 plasmids have good response to rhIL-11, so such cells could be used for subsequent stable transformation screening.

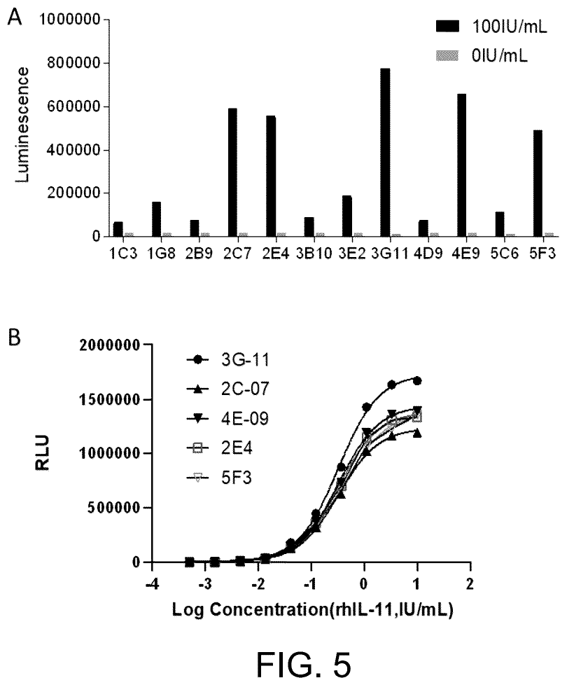

2-2) Stable transfection cell testing: After transfection of pPR(EXP)-STAT3 plasmids into the 293T cells, stably transfected mixed clone 293T-STAT3 cells were obtained by pressure screening with 0.5 μg/mL puromycin for 4 weeks. The reactivity to rhIL-11 was tested. According to a reaction curve of transient transfected cells, the pre-dilution concentration of rhIL-11 was adjusted to 333.3 IU/mL. Results are shown in FIG. 4. The chemiluminescence intensity reaches the maximum under the stimulation of rhIL-11 at 37.03 IU/mL. EC₅₀ is 0.7661, R² is 0.994, and reliability of experimental results is high. The experiment proves that the constructed pPR(EXP)-STAT3 plasmids have better response to rhIL-11 after stably transfected into the 293T cells. Such cells can be used for screening monoclonal cells.
2-3) Monoclonal cell isolation: The cell density of the stable mixed clone 293-STAT3 cells was adjusted to 5 cells/mL by a limiting dilution method. At the condition of 0.5 μg/mL puromycin as a screening concentration, screening was performed for 4 successive weeks, and 12 monoclonal cell strains were obtained. According to the stably transfected cell reaction curve, rhIL-11 at 100 IU/mL was selected to stimulate the labeled monoclonal cells, and the reactivity of the monoclonal cells to rhIL-11 was tested. As shown in A of FIG. 5, 5 monoclonal cell strains namely 2C7, 2E4, 3G11, 4E9, and 5F3 had higher chemiluminescence intensities under the action of rhIL-11,and 3G11 had the highest chemiluminescence intensity. These 5 cell lines were stimulated with rhIL-11, and four-parameter curves were drawn according to the luminescence intensities at different concentrations. As shown in B of FIG. 5, these 5 cell strains all had good dose-response relationship to rhIL-11, and R² was greater than 0.99, indicating that the experimental data had high reliability. Among the cell strains, 3G11 had the highest maximum chemiluminescence value, the highest signal-to-noise ratio and the most sensitive response. The maximum chemiluminescence value, signal-to-noise ratio and four-parameter curve shapes of other 4 cell lines were similar. Therefore, in the present disclosure, the 3G11 monoclonal cell strain named as 293T-3G11 was selected for establishing rhIL-11 reporter gene method methodology.

### Example 3: Signaling Pathway Verification

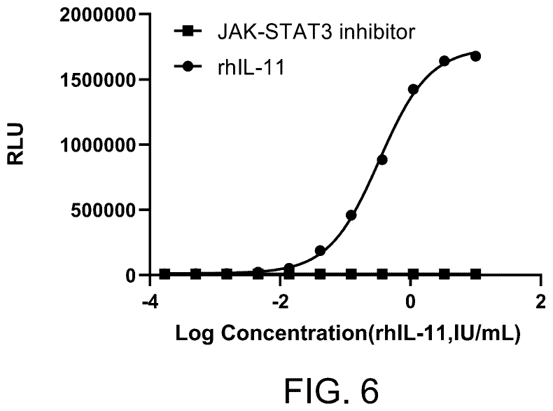

To verify whether the signaling pathway of cells satisfy an expectation, the 293T-G11 cell strain was stimulated jointly by rhIL-11 and a JAK-STAT3 pathway inhibitor, and chemiluminescence intensities at different concentration points of rhIL-11 was tested and compared with those of the 293T-G11 cell strain stimulated by rhIL-11. A four-parameter curve was fitted. As shown in FIG. 6, the JAK-STAT3 pathway inhibitor can inhibit expression of luciferase genes in the 293T-G11 cells, and the chemiluminescence intensity is low, while the cells without the inhibitor can normally express luciferase. A four-parameter curve can be drawn according to the chemiluminescence intensity. The experiment shows that the construction of the signaling pathway in the cells satisfy the expectation, and the 293T-G11 cell strain is suitable for subsequent experiments.

### Example 4: Method Optimization of IL-11 Reporter Gene Method

1. Optimization of rhIL-11 pre-dilution factor and dilution gradient: rhIL-11 was pre-diluted to 100 IU/mL on the basis of stable transfection results. 14 gradients of rhIL-11 diluted by 2 folds, 3 folds and 4 folds in the complete medium were prepared in total, with 3 duplicate wells for each concentration. For other conditions, see Examples 1-4. The chemiluminescence intensities at different dilution folds were tested, four-parameter curves were fitted, signal-to-noise ratios were compared, and the most appropriate dilution fold was selected as the subsequent experimental condition.
2. Optimization of inoculation quantity: Cells in the logarithmic phase were collected, the cell concentration was adjusted with the complete medium, and the cells were inoculated into 96-well plates with different cell densities of various plates, namely 4×10⁴ cells/well, 5×10⁴ cells/well, 6×10⁴ cells/well, 7×10⁴ cells/well and 8×10⁴ cells/well respectively. See Examples 1-4 for other conditions and operation procedures. Chemiluminescence intensities of the inoculation quantity of cells, four-parameter curves were fitted, and signal-to-noise ratios were compared to select the most suitable inoculation quantity of cells as the subsequent experimental condition.
3. Optimization of rhIL-11 incubation time: The cells with adjusted concentrations were inoculated into four 96-well plates, and other conditions were described in Examples 1-4. After 3 h of action, the first plate was taken out to test the expression of luciferase. Thereafter, one plate was taken out every 1 h to test the expression of luciferase. The chemiluminescence intensities of rhIL-11 after incubation for 3, 4, 5 and 6 h were tested respectively. Four-parameter curves were fitted, and signal-to-noise ratios were compared to select the optimal incubation time as the

### Optimization Results of IL-11 Reporter Gene Method

1. Optimization of rhIL-11 pre-dilution factor and dilution gradient

In order to test the reactivity of the 293T-G11 cells isolated from the monoclonal cells to rhIL-11, rhIL-11 is pre-diluted to 100 IU/mL and diluted by 4 folds to stimulate the 293T-G11 cells in this study. The shape of the observed four-parameter curve is good, R² is greater than 0.99, and the experimental reliability is high.

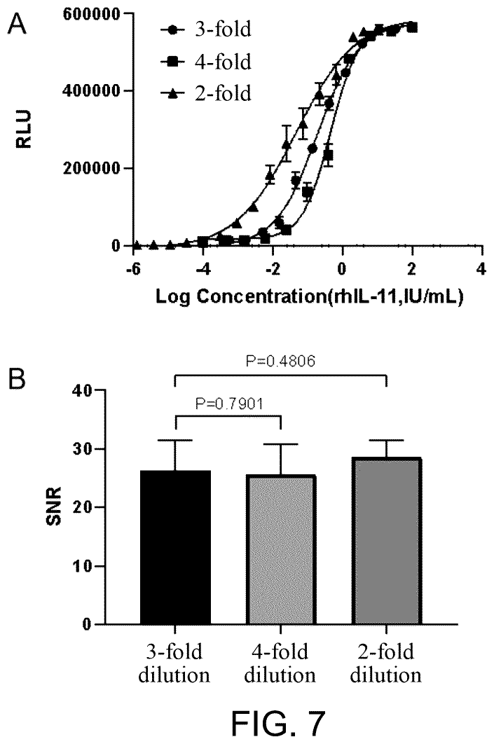

2-fold, 3-fold and 4-fold serial dilutions are validated. The signal-to-noise ratios are shown in B of FIG. 7. In 2-fold, 3-fold and 4-fold serial dilutions, the signal-to-noise ratios are greater than 20, indicating that the reactivity of rhIL-11 tested by RGA is good, and there is no significant difference (P>0.05), indicating that the cell reactivity is good under the three dilution gradients. Analysis results are shown in A of FIG. 7. The EC₅₀ of 2-fold dilution is 0.04628, and R² is 0.9902. The EC₅₀ of 4-fold dilution is 0.4545, and R² is 0.9926. The EC₅₀ of 3-fold dilution is 0.1901, and R² is 0.9966. All R² is greater than 0.99, indicating that the experimental data have high reliability. In the four-parameter curve of 3-fold dilution, points are evenly distributed, an upper platform and a lower platform each are provided with two points, and four points are arranged on an oblique line, such that the four-parameter curve covers the whole influence process of dose change on the chemiluminescence intensity. Based on the result, the present disclosure selects the pre-dilution concentration of 20 IU/mL, the 3-fold dilution and eight gradients as the optimized RGA testing conditions.

### 2. Optimization of Inoculation Quantity of Cells

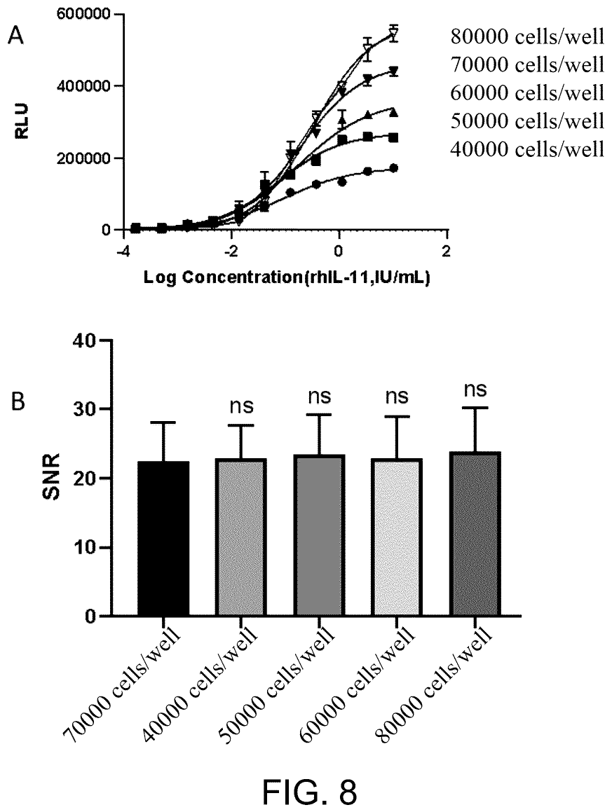

In the present disclosure, luminescence intensities of rhIL-11 with different inoculation quantities of cells are tested, and results are shown in A of FIG. 8. It is found in the present disclosure that under the stimulation of rhIL-11, the four-parameter curves under different inoculation quantities of cells have similar shapes, and the upper platforms are basically parallel, indicating that the influence of the inoculation quantity of cells on rhIL-11 activity testing results tends to be stable. When the cell quantity is 40000 cells/well, the EC₅₀ is 0.1757. When the cell quantity is 50000 cells/well, the EC₅₀ is 0.1476. When the cell quantity is 60000 cells/well, the EC₅₀ is 0.4524. When the cell quantity is 70000 cells/well, the EC₅₀ is 0.4278. When the cell quantity is 80000 cells/well, the EC₅₀ is 0.4241. All R² is greater than 0.99, indicating high reliability of the experimental data. The experimental results show that when the quantity of cells per well reaches more than 60000, values of EC₅₀ are basically consistent, indicating that an increase of the quantity of cells per well tends to have consistent effects on RGA testing results. The signal-to-noise ratio analysis is shown in B of FIG. 8, and the signal-to-noise ratios under different inoculation quantities of cells are greater than 20, indicating that the reactivity of RGA for testing rhIL-11 is good under different cell quantities. Data analysis shows that there is no significant difference in signal-to-noise ratios under different cell quantities (P>0.05). In the case of 80000 cells per well, excessive cell quantity may lead to excessive chemiluminescence intensity, which may lead to overexposure and increase errors. In order to ensure the stability of the experiment, the inoculation quantity of 70000 cells per well can satisfy the experimental requirements, and therefore, the present disclosure decides to employ this inoculation quantity in subsequent testing.

### 3. Optimization of RhIL-11 Incubation Time

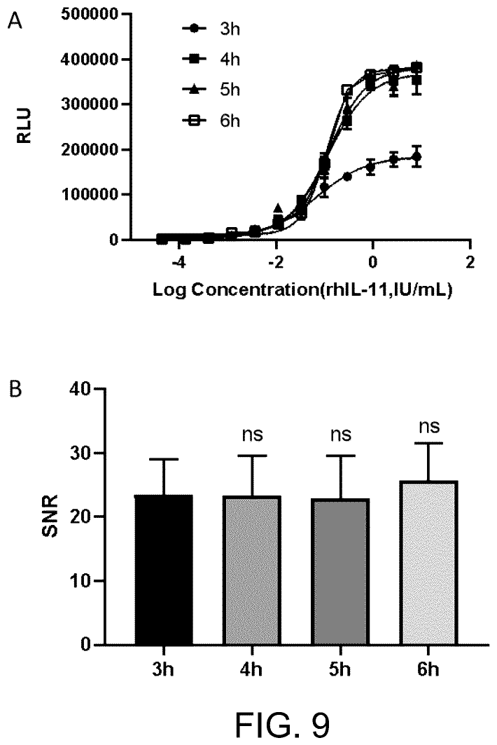

The chemiluminescence intensities of rhIL-11 at 3 h, 4 h, 5 h and 6 h after incubation of 293T-G11 are tested respectively, and four-parameter curves were fitted. The results are shown in A of FIG. 9. When the drug incubation time is 3 h, the EC₅₀ is 0.06595, and R² is 0.9808. When the incubation time is 4 h, the EC₅₀ is 0.1150, and R² is 0.9894. When the incubation time is 5 h, the EC₅₀ is 0.1208, and R² is 0.9833. When the incubation time is 6 h, the EC₅₀ is 0.1103, and R² is 0.9954. After the incubation time reaches 4 h, values of EC₅₀ are basically consistent, indicating that after the incubation time reaches 4 h, the effects of prolonged incubation time on RGA results tend to be consistent, and all R² is greater than 0.99, indicating that the experimental data have high reliability. The signal-to-noise ratios are shown in B of FIG. 9. The signal-to-noise ratios are greater than 20 at different incubation time, indicating that the reactivity of rhIL-11 tested by RGA is good at different incubation time, and there is no significant difference (P>0.05). Experiments show that the four-parameter curves are basically coincident after the drug action time reaches 4 h, indicating that the incubation time of 4 h has already satisfied the testing requirements, and an increase of the incubation time cannot produce a greater chemiluminescence intensity. In consideration of saving time and cost, 4 h is selected as the optimized drug incubation time, and 4 h is used as the testing condition in the subsequent RGA testing.

### 4. Optimization Results of Key Parameters and Specific Operation Procedures

See Table 1 for optimization results of key parameters for testing the biological activity of rhIL-11 by RGA.

TABLE 1

| Parameter item | Parameter value | |
|---|---|---|
| Number of cells | 70000 | cells/well |
| Culture time | 17-24 | h |
| Pre-dilution concentration | 20 | IU/mL |
| Fold-dilution factor | 3 | fold |
| Incubation time | 4 | h |

The optimized operation procedures for rhIL-11 activity determination by RGA:
1) collect 293T-G11 cells in the logarithmic growth phase;
2) inoculate cells into 96-well plates with 70000 cells per well, and perform culture at 37° C. and 5% CO₂ for 17-24 h;
3) dissolve rhIL-11 samples in water for injection, adjust the samples to 20 IU/mL with a complete medium, perform 3-fold dilution with 10 gradients, make 100 μL for per well for diluted rhIL-11 samples, make 3 replicate wells in parallel, and perform incubation for 4 h; and
4) test the luminescence intensity by using a multifunctional microplate reader according to the operation of the luciferase assay kit.

### Methodology Validation of IL-11 Reporter Gene Method

1. Accuracy: Two rhIL-11 samples were taken and each were dissolved in 1 mL of water for injection. The sample was diluted to 20 IU/mL according to the labeled value. One rhIL-11 reference substance was taken and dissolved in water for injection, and then was diluted to 20 IU/mL. The pre-diluted sample and the reference substance were mixed at 1:1, and then testing was performed according to the optimized experimental operation.

The biological activity of the sample was calculated according to the formula:

$$biological activity of sample for testing ( \frac{IU}{mL} ) = Pr \times \frac{Ds \times Es}{Dr \times Er}$$

In the formula, Pr represent the biological activity of a standard substance with unit of IU/ml, Ds represents the pre-dilution factor of the test sample, and Drrepresent a pre-dilution factor of the standard substance. Es represents a dilution factor of the test sample equivalent to the half effective dose of the standard substance, and Er represents a dilution factor of the half effective dose of the standard substance.

$$recovery rate ( \% ) = \frac{measured biological activity of sample rhIL - 11}{theoretical biological activity value of sample rhIL - 11} \times 100 \%$$

### 2. Precision

Within-day precision: Two rhIL-11 samples were taken and each were dissolved in 1 mL of sterile water for injection. The activity of two batches of samples was tested according to the optimized experimental operation. Each batch of samples was tested 6 times, with 3 duplicate wells in parallel, so as to evaluate the within-day precision.

Inter-day precision: Two rhIL-11 samples were taken and each were dissolved in 1 mL of sterile water for injection. The activity of two batches of samples was tested according to the optimized experimental operation. Each batch of samples was tested 6 times, with 3 duplicate wells in parallel, and parallel testing was performed for 3 d. Inter-day precision was evaluated.

### 3. Specificity

Specificity of cells to denatured rhIL-11: Two rhIL-11 samples were taken, each were dissolved in 1 mL of sterile water for injection, and were heated at 70° C. for 2 h and 10 h respectively. The activity of rhIL-11 and normal rhIL-11 was tested according to the optimized experimental operation, and the relative biological activity of denatured rhIL-11 was calculated. Relative biological activity=EC₅₀ of sample/EC₅₀ of reference substance×100%.

Specificity of cells to different cytokines: A variety of different cytokines were selected: IL-2, IL-6, IL-21, IL-31, IL-18, IFNα-1, rhEGP, rhEPO, CT-1, and rhGM-CSF. These cytokines were dissolved in 1 mL of water for injection, and pre-diluted to 20 IU/mL according to labeled values. The activity of different cytokines was tested.

### 4. Stability

Stability of 293T-G11 cells in selective medium: 293T-G11 cells of different generations (generation 10, generation 20 and generation 40) under selective pressure of puromycin were taken, the activity of rhIL-11 in two batches was tested, and each sample was tested 3 times.

Stability of 293T-G11 cells in growth medium: 293T-G11 cells of different generations (generation 10, generation 20 and generation 40) without selective pressure were taken, the activity of rhIL-11 in two batches was tested, and each sample was tested 3 times.
5. Robustness: Verification was achieved by observing whether biological activity testing results of rhIL-11 are affected by slightly changing experimental conditions, and the changed conditions were as follows:

Incubation time: Incubation time was adjusted to 4 h-5 h with once every half hour.

Luciferase substrate amount: The luciferase substrate amount was adjusted to 90 μL-110 μL with an interval of 10 μL.

Luminescence time: Luminescence time was adjusted to 1 min, 10 min and 30 min.
6. Applicability: The biological activity of rhIL-11 was tested by replacing luciferase substrates with Bio-Glo, Steady-Glo, and Bright-Glo.
7. Comparison with proliferation inhibition method: The relative biological activity of 6 groups of different rhIL-11 samples was determined by a pharmacopoeia method and RGA respectively, and parallel determination was made 3 times.

### Methodology Validation Results of IL-11 Reporter Gene Method

Accuracy: the method provided by the present disclosure perform testing by using a recovery rate of an added standard substance. According to the present disclosure, two rhIL-11 samples diluted in advance each are mixed with a reference substance according to the ratio of 1:1, and effective determination is carried out 6 times according to the specific operation procedures of biological activity determination of rhIL-11. Results are listed in Table 2. The maximum recovery rate of sample 1 is 106.81%, the minimum recovery rate is 88.89%, and a coefficient of variation (CV) is 6.62%. The maximum recovery rate of sample 2 is 112.38%, the minimum recovery rate is 99.21%, and the CV is 4.28%. The recoveries of the two batches of samples are between 80-120%, indicating that the activity of the samples measured by RGA is close to the labeled value of the samples, and the accuracy is good. Values of the CV of the two batches of samples are less than 10%, indicating that the accuracy of RGA in testing rhIL-11 activity is high.

TABLE 2
Experimental results of accuracy of reporter
gene method for testing activity of IL-11

| | Sample 1 | Recovery | Sample 2 | Recovery |
|---|---|---|---|---|
| | (IU/mL) | rate | (IU/mL) | rate |
| 1 | 741264.9497 | 92.66% | 824335.9059 | 103.04% |
| 2 | 711131.9094 | 88.89% | 824922.7292 | 103.12% |
| 3 | 760509.5894 | 95.06% | 899045.6545 | 112.38% |
| 4 | 765747.3777 | 95.72% | 855616.5999 | 106.95% |
| 5 | 854512.4364 | 106.81% | 829168.3237 | 103.65% |
| 6 | 811399.8594 | 101.42% | 793680.992 | 99.21% |
| Mean | 774094.3537 | — | 837795.0342 | — |
| (IU/mL) | | | | |
| CV | 6.62 | — | 4.28 | — |

### Precision

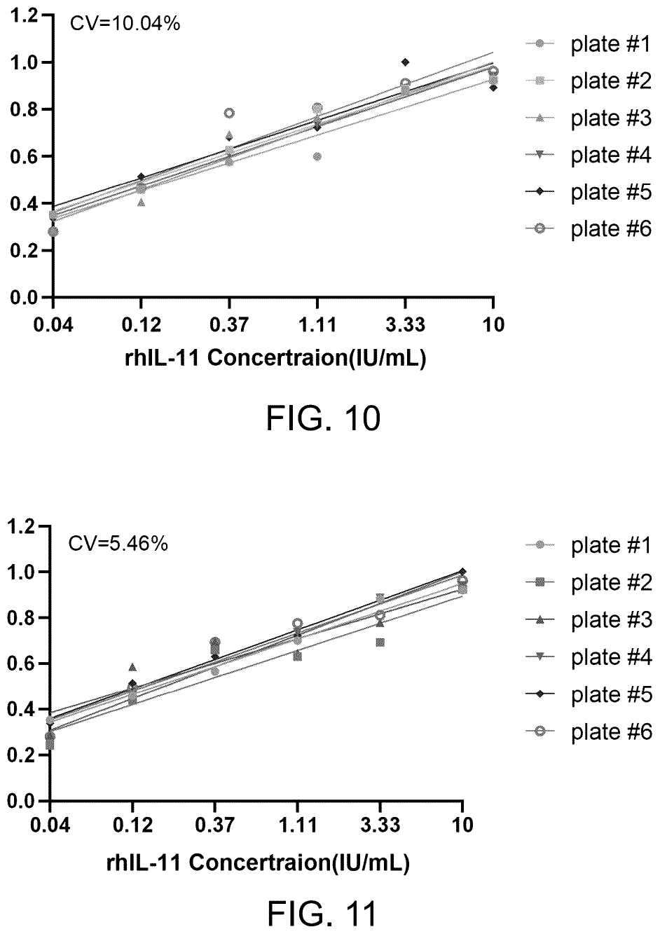

Within-day precision: According to the optimized parameters, the activity of two batches of rhIL-11 samples is tested with 6 times for each batch and 3 wells for each time. The within—day precision of RGA is verified, and results are shown in Table 3. The maximum recovery rate of sample 1 is 109.35%, and the minimum recovery rate is 84.68%. The maximum recovery rate of sample 2 is 106.15%, and the minimum recovery rate is 96.71%. The recovery rates of the two batches of samples are between 80-120%, indicating that the sample activity measured by RGA is close to the labeled value of the sample, indicating good accuracy. The coefficient of variation (CV) of sample 1 is 10.04%, and the coefficient of variation (CV) of sample 2 is 5.46%. The ratio of the luminescence value of each well of each batch of samples to the maximum luminescence value of this batch is calculated and plotting is made (FIGS. 10 and 11). Values of the CV of 6 batches tested within one day are all less than 10%, indicating that the within-day precision of RGA in testing activity of rhIL-11 is good.

TABLE 3
Experimental results of within-day precision of reporter
gene method for testing activity of IL-11

| | Sample 1 | Recovery | Sample 2 | Recovery |
|---|---|---|---|---|
| | (IU/mL) | rate | (IU/mL) | rate |
| 1 | 710405.19 | 88.80% | 773679.09 | 96.71% |
| 2 | 704051.86 | 88.01% | 849205.83 | 106.15% |
| 3 | 714619.12 | 89.33% | 811766.61 | 101.47% |
| 4 | 688492.71 | 86.06% | 774720.67 | 96.84% |
| 5 | 677471.64 | 84.68% | 820724.34 | 102.59% |
| 6 | 874813.61 | 109.35% | 726776.44 | 90.85% |
| Mean | 728309.02 | — | 792812.17 | — |
| (IU/mL) | | | | |
| CV | 10.04% | — | 5.46% | — |

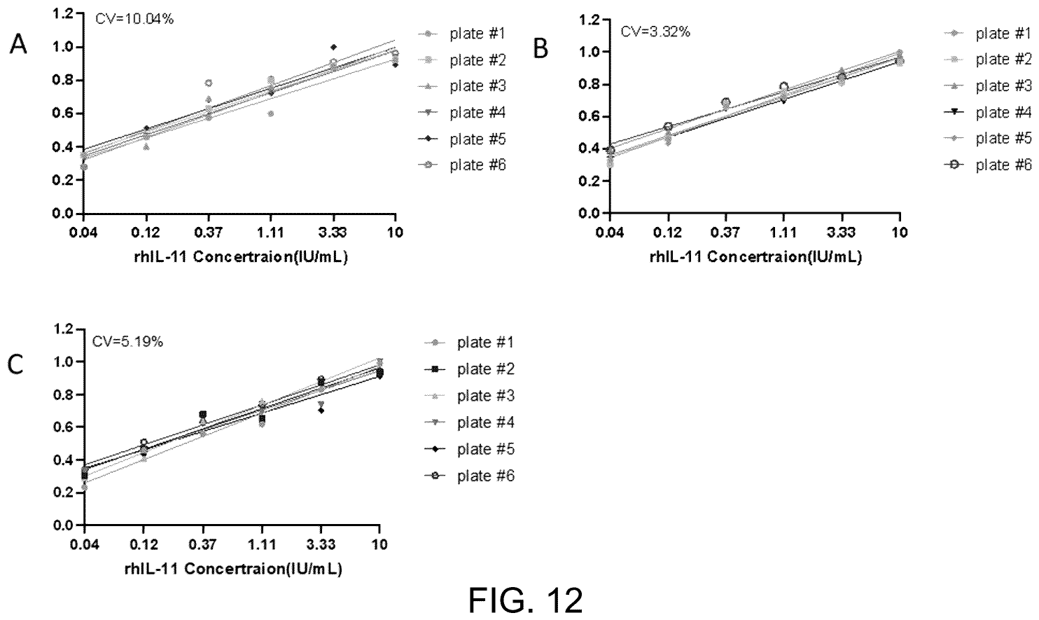

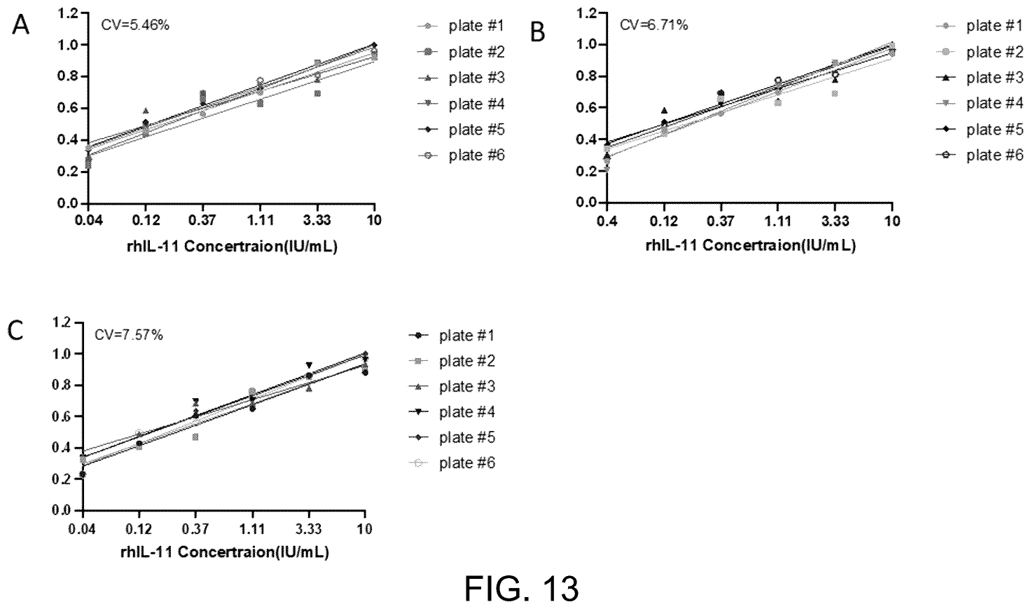

Inter-day precision: According to the optimized parameters, the activity of two batches of rhIL-11 samples is tested with 6 times for each batch and 3 duplicate wells, and parallel testing is performed for 3 d. The inter-day precision of the method is evaluated by analyzing the coefficient of variation of each sample within 3 days, and results are shown in Table 4 and Table 5. The average recovery rate ranges of sample 1 are: (84.68-109.35) %, (96.46-103.01) %, and (91.71-105.91) %, and the range is (80-120) %, indicating good accuracy. The average recovery rate ranges of sample 2 are: (96.71-106.15) %, (91.03-106.39) % and (87.78-107.45) %, and the rage is (80-120) %, indicating good accuracy. The coefficients of variation (CV) of sample 1 are 10.04%, 3.32%, and 5.19%, and the coefficients of variation (CV) of sample 2 are 5.46%, 6.71%, and 7.57%. The ratio of the luminescence value of each well of each batch of samples to the maximum luminescence value of this batch is calculated, and plotting is made (FIGS. 12 and 13), indicating that the inter-day precision of this method is good.

TABLE 4
Experimental results of inter-day precision of reporter
gene method for testing activity of rhIL-11 (sample 1)

| | Sample of day 1 | Recovery | Sample of day 2 | Recovery | Sample of day | Recovery |
|---|---|---|---|---|---|---|
| | (IU/mL) | rate | (IU/mL) | rate | 3 (IU/mL) | rate |
| 1 | 710405.19 | 88.80% | 824042.14 | 103.01% | 733678.13 | 91.71% |
| 2 | 704051.86 | 88.01% | 784820.85 | 98.10% | 778664.67 | 97.33% |
| 3 | 714619.12 | 89.33% | 803180.87 | 100.40% | 780506.71 | 97.56% |
| 4 | 688492.71 | 86.06% | 771644.69 | 96.46% | 833806.69 | 104.23% |
| 5 | 677471.63 | 84.68% | 793007.99 | 99.13% | 791178.00 | 98.90% |
| 6 | 874813.61 | 109.35% | 802261.03 | 100.28% | 847275.14 | 105.91% |
| Mean | 728309.02 | — | 760560.59 | | 794184.89 | — |
| (IU/mL) | | | | | | |
| CV | 10.04% | — | 3.32% | — | 5.19% | — |

TABLE 5
Experimental results of inter-day precision of reporter
gene method for testing activity of rhIL-11 (sample 2)

| | | | | | Sample of | |
|---|---|---|---|---|---|---|
| | Sample of day 1 | Recovery | Sample of day 2 | Recovery | day 3 | Recovery |
| | (IU/mL) | rate | (IU/mL) | rate | (IU/mL) | rate |
| 1 | 773679.09 | 96.71% | 731872.31 | 91.48% | 737918.61 | 92.24% |
| 2 | 849205.83 | 106.15% | 824512.71 | 103.06% | 797033.07 | 99.63% |
| 3 | 811766.61 | 101.47% | 811267.20 | 101.41% | 820113.39 | 102.51% |
| 4 | 774720.67 | 96.84% | 851138.98 | 106.39% | 859620.02 | 107.45% |
| 5 | 820724.34 | 102.59% | 728253.78 | 91.03% | 702204.48 | 87.78% |
| 6 | 726776.44 | 90.85% | 751192.22 | 93.90% | 831388.42 | 103.92% |
| Mean | 792812.17 | — | 783039.53 | | 791379.67 | — |
| (IU/mL) | | | | | | |
| CV | 5.46% | — | 6.71% | — | 7.57% | |

### Specificity

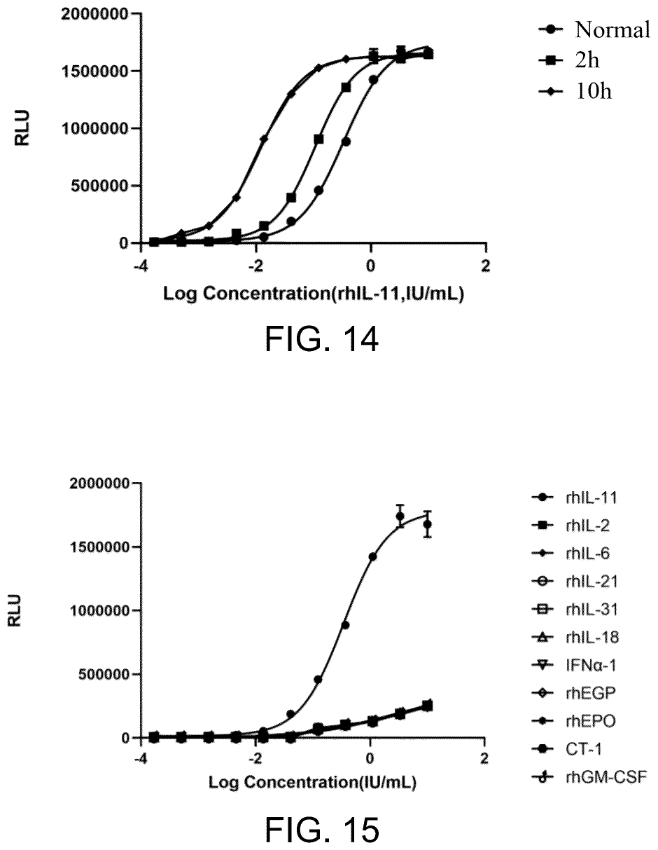

Specificity of cells to denatured rhIL-11: The activity of rhIL-11 and normal rhIL-11 is tested according to the optimized parameters, and the relative biological activity of denatured rhIL-11 is calculated. Results are shown in FIG. 14, the EC₅₀ of the normal sample is 0.3334, and R² is 0.9977. The EC₅₀ of the sample heated at 70° C. for 2 h is 0.1076, and R² is 0.9983. The EC₅₀ of the sample heated at 70° C. for 10 h is 0.01206, and R² is 0.9984. The relative biological activity of rhIL-11 heated at 70° C. for 2 h and 10 h is 32.27% and 3.62% respectively, and the relative biological activity is decreased. However, the maximum chemiluminescence intensity is not changed, because although rhIL-11 is denatured by heat, some protein structures can activate the cellular pathway of 293T-G11 to produce chemiluminescence. Therefore, this method can also be used for evaluating the biological activity of denatured rhIL-11 and stability of rhIL-11.

Specificity of cells for antibodies to different targets: According to the optimized parameters, biological activity of rhIL-111 and 10 different cytokines is tested. As shown in FIG. 15, only rhIL-11 can stimulate luciferase expression of 293-G11 cells. According to the luminescence intensity, a four-parameter curve is fitted, the EC₅₀ value is 0.3459, and R² is 0.9943, which proves that the reliability of the experimental data is high. Meanwhile, other 10 cytokines cannot make luciferase of 293T-G11 be expressed, and chemiluminescence intensity is almost non-existent. When the concentrations of the cytokines are high, a little chemiluminescence will be caused, probably because the selected cytokines all act on the JAK-STAT pathway, and there is a corresponding receptor on the cell surface. When the concentration of the corresponding cytokine is high, the signaling pathway is activated under binding with the cell surface receptor, causing trace chemiluminescence, but the chemiluminescence is different from the chemiluminescence produced by rhIL-11 by two orders of magnitude, which can be ignored. In summary, this method can specifically test the biological activity of rhIL-11.

### Stability

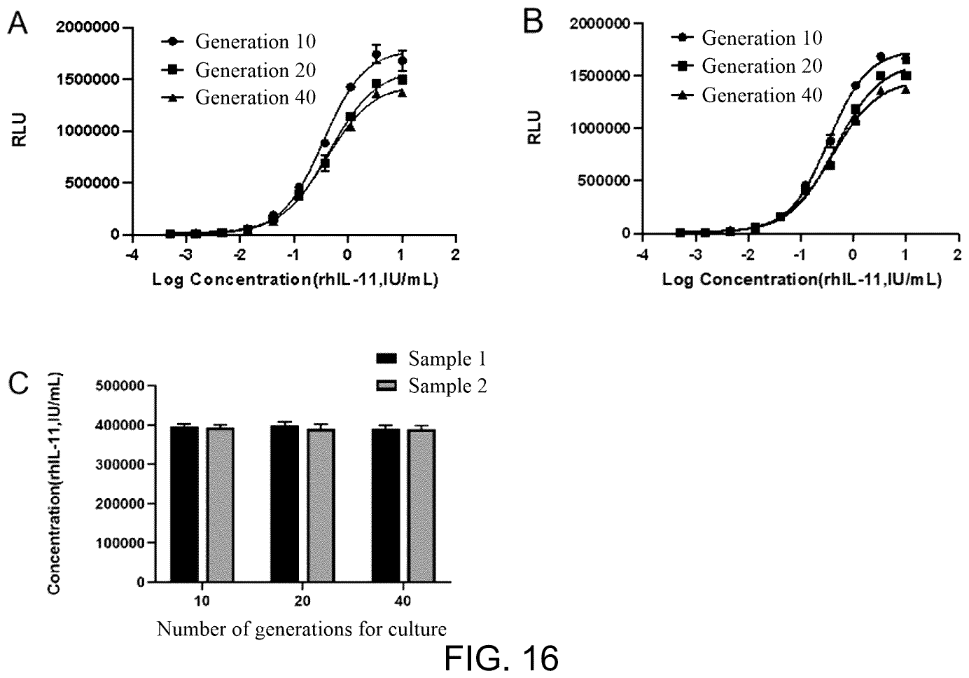

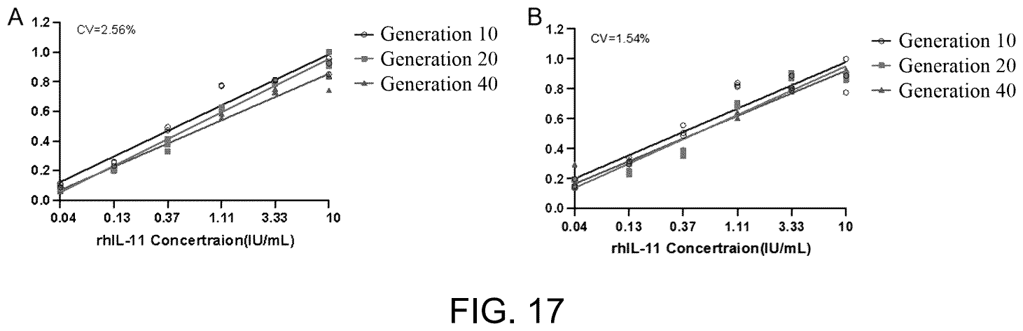

Stability of 293T-G11 cells in selective medium: 293T-G11 cells of different generations (generation 10, generation 20 and generation 40) under selective pressure of puromycin were taken, and the activity of rhIL-11 in two batches was tested. After the two batches of samples act on the 293T-G11 cells, four-parameter curves are fitted according to the chemiluminescence intensities (A and B in FIG. 16). The maximum luminescence intensity and the dose-response curve shape of four-parameter fitting are basically the same. Values of the EC₅₀ of different generations of cells of sample 1 are 0.3466, 0.4489 and 0.3905 respectively, values of the EC₅₀ are basically the same, and all R² is greater than 0.99, indicating that the reliability of data is high. Values of the EC₅₀ of different generations of cells of sample 2 are 0.3832, 0.461 and 0.3977 respectively, values of the EC₅₀ are basically the same, and all R² is greater than 0.99, indicating that the reliability of data is high. The ratio of the luminescence value of each well of each batch of samples to the maximum luminescence value of this batch is calculated, and plotting is made (FIG. 17). The activity values of samples tested with cells of different generations are shown in Table 6. The CV of sample 1 is 2.56%, the CV of sample 2 is 1.54%, and both the values are less than 10%. After one-way analysis of variance, results show no statistical difference (P>0.05), indicating that there is no statistical difference between the testing results of different generations. These results indicate that the generation number of cells has no significant effect on the biological activity of rhIL-11 tested by the cells when the cells are continuously passaged to generation 40,which confirms that the cell strain can be stably passaged under the selective pressure of puromycin.

TABLE 6
Activity testing of samples with different
generations of cells in selective medium

| Selective | Generation | Generation | Generation |
|---|---|---|---|
| medium | 10 | 20 | 40 |
| Sample 1 | 807371.7272 | 774391.4076 | 780479.5584 |
| | 811540.6524 | 786887.7496 | 783897.0936 |
| | 787808.2672 | 786239.069 | 793706.2528 |
| Sample 2 | 762683.919 | 815562.9188 | 806175.3892 |
| | 777174.9698 | 792323.9494 | 815740.2566 |
| | 790830.4634 | 762231.7782 | 788644.0918 |

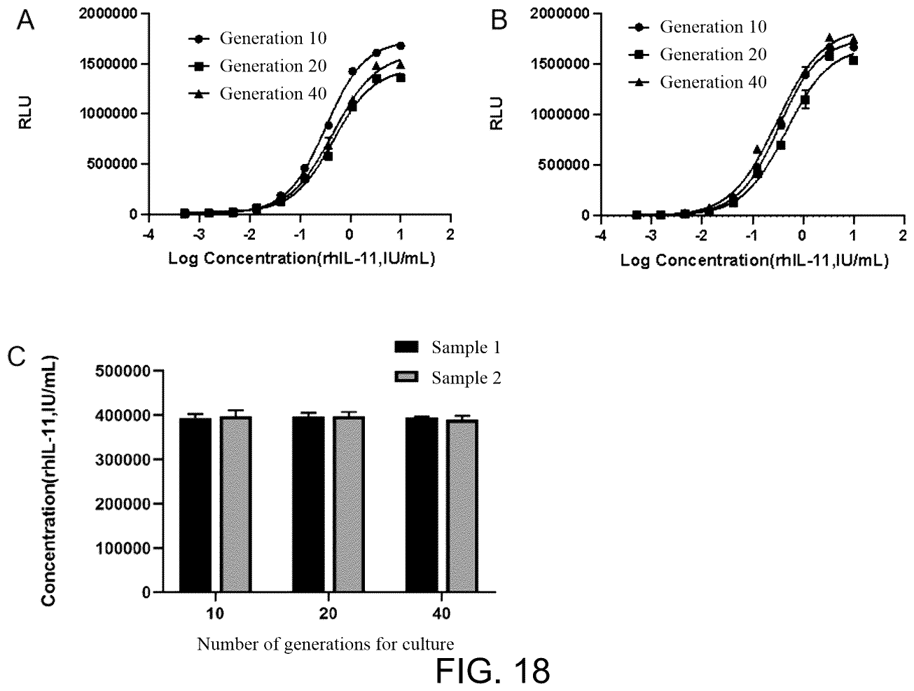

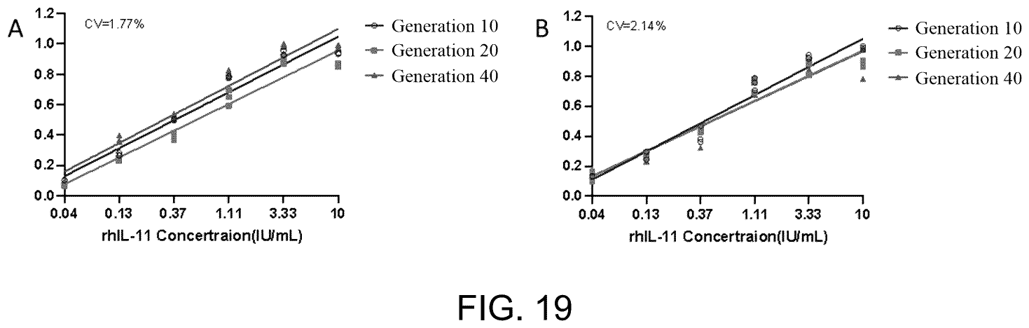

Stability of 293T-G11 cells in growth medium: Two batches of rhIL-11 samples are tested when the cells were continuously passaged to generation 40 in the growth medium after removal of selective pressure. After these two batches of samples act on 293T-G11 cells respectively, four-parameter curves are fitted (A and B of FIG. 18). The maximum luminescence intensity and the dose-response curve shape of four-parameter fitting are basically the same. The EC₅₀ values of different generations of cells of sample 1 are 0.3279, 0.4625 and 0.4491 respectively, the EC₅₀ values are basically the same, and all R₂ values are greater than 0.99, indicating that the reliability of data is high. The EC₅₀ values of different generations of cells of sample 2 are 0.3581, 0.4419 and 0.3862 respectively, the EC₅₀ values are basically the same, and all R² is greater than 0.99, indicating that the reliability of data is high. The ratio of the luminescence value of each well of each batch of samples to the maximum luminescence value of this batch is calculated, and FIG. 19 is plotted. The activity values of samples obtained through testing with cells of different generations are shown in Table 7. The CV of sample 1 is 1.77%, the CV of sample 2 is 2.14%, and both the values are less than 10%. Through one-way analysis of variance, the results show no statistical difference (P>0.05) (see Appendix B), which indicates that the target plasmid is integrated into the genome of the cells, and the stable passaging can be achieved after removal of the selective pressure of puromycin. The target gene will not be lost due to passaging, and in short, the cell line is stable. According to the above results, the cells constructed by this method perform well in generation stability.

TABLE 7
Activity testing of samples with different
generations of cells in growth medium

| Growth | Generation | Generation | Generation |
|---|---|---|---|
| medium | 10 | 20 | 40 |
| Sample 1 | 797207.9608 | 779720.7778 | 801282.5298 |
| | 788220.7052 | 817167.3898 | 786687.4412 |
| | 769631.381 | 799245.3192 | 782905.7466 |
| Sample 2 | 786829.4068 | 770933.2168 | 801377.3092 |
| | 805906.2898 | 767441.0392 | 769998.921 |
| | 772971.5592 | 798575.1576 | 761071.524 |

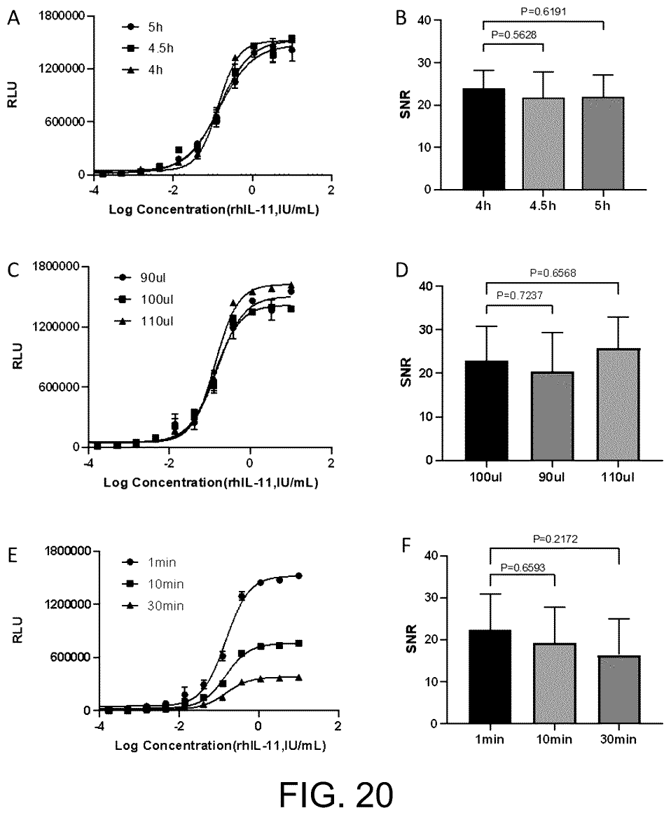

Robustness: According to the optimized parameters, testing is performed, and when the incubation time of rhIL-11 changes by 0.5-1 h, four-parameter curves are fitted according to the chemiluminescence intensity, as shown in A and B of FIG. 20. When the incubation time changes slightly, the maximum chemiluminescence value is basically consistent with the four-parameter curve shape. The EC₅₀ is 0.1378 when the inoculation time is 4 h, the EC₅₀ is 0.1510 when the inoculation time is 4.5 h, and the EC50 is 0.1438 when the inoculation time is 5 h. The EC₅₀ values are relatively consistent, all R² is greater than 0.99, and signal-to-noise ratios are greater than 20, indicating that RGA has good reactivity in testing rhIL-11 when the incubation time is slightly changed. According to analysis, when the incubation time is slightly changed, P=0.5628 for the signal-to-noise ratios when the inoculation time is 4 h and 4.5 h respectively, P=0.6191 for the signal-to-noise ratios when the inoculation time is 4 h and 5 h respectively, and P values are greater than 0.05, so there is no statistical difference between the signal-to-noise ratios.

When the luciferase substrate amount is changed by up to 10 μL, four-parameter curves are fitted according to the chemiluminescence intensity as shown in C and D of FIG. 20. When the luciferase substrate amount is changed slightly, the maximum chemiluminescence value is basically consistent with the four-parameter curve. The EC₅₀ at the amount of 90 μL is 0.1496, the EC₅₀ at the amount of 100 μL is 0.1290, and the EC₅₀ at the amount of 110 μL is 0.1349. The EC₅₀ values are relatively consistent, all R² is greater than 0.99, and signal-to-noise ratios are greater than 20, indicating that RGA has good reactivity in testing rhIL-11 when the luciferase substrate amount is slightly changed. Through analysis, when the luciferase substrate amount is slightly changed, P=0.7237 for the signal-to-noise ratios when the substrate amounts are 90 μL and 100 μL respectively, P=0.6568 for the signal-to-noise ratios when the substrate amounts are 90 μL and 110 μL respectively, and P values are all greater than 0.05, so there is no statistical difference between the signal-to-noise ratios.

When the action time of the luciferase substrate amount is changed, four-parameter curves are fitted according to the chemiluminescence intensity as shown in E and F of FIG. 20. The chemiluminescence value is gradually decreased with the longer luminescence time, and EC₅₀ values are relatively consistent. The EC₅₀ is 0.1466 when the action time is 1 min, the EC₅₀ is 0.1449 when the action time is 10 min, and the EC₅₀ is 0.1434 when the action time is 30 min. All R² is greater than 0.99, the signal-to-noise ratio is decreased, but there is still no statistical difference. P=6593 for the signal-to-noise ratios when the action time is 1 min and 10 min respectively, P=2172 for the signal-to-noise ratios when the action time is 1 min and 30 min respectively, and P values are greater than 0.05, so there is no statistical difference between the signal-to-noise ratios.

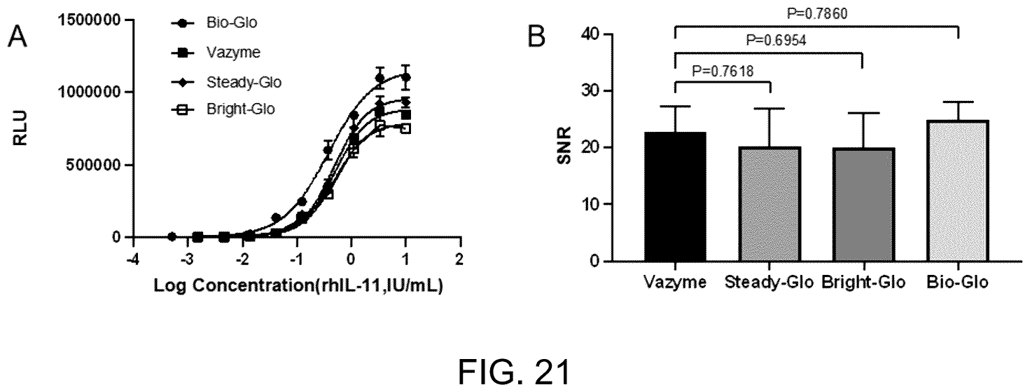

Applicability: Testing is performed according to the optimized parameters, other conditions keep unchanged, and luciferase substrates are replaced with Bio-Glo, Steady-Glo, and Bright-Glo. A batch of rhIL-11 samples are tested for activity respectively with 3 duplicate wells in parallel. Four-parameter curves are fitted according to the chemiluminescence intensity. Results are shown in A of FIG. 21. It can be seen from the figure that different luciferase substrates can produce the good dose effect. It can be found that there is no significant difference between values of the EC₅₀ of the four luciferase substrates. The EC₅₀ of the luciferase assay kit of Vazyme is 0.4937, the EC₅₀ of Steady-Glo is 0.4843, the EC₅₀ of Bright-Glo is 0.4883, and the EC₅₀ of Bio-Glo is 0.3786. There is no significant difference among the values of the EC₅₀ of the four kinds of luciferase. All R² is greater than 0.99, and therefore, the reliability of data is high. The signal-to-noise ratios of different luciferase substrates are greater than 20, indicating that RGA has good reactivity in testing rhIL-11. Through analysis, when the luciferase substrate amount is slightly changed, P=0.7618 for the signal-to-noise ratios of Vazyme and Steady-Glo, P=0.6954 for the signal-to-noise ratios of Vazyme and Bright-Glo, P=0.7860 for the signal-to-noise ratios of Vazyme and Bio-Glo, and P values are all greater than 0.05, so there is no statistical difference between the signal-to-noise ratios.

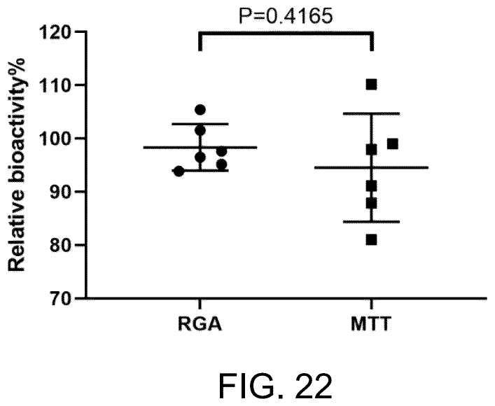

Comparison with proliferation inhibition method: The biological activity of rhIL-11 is simultaneously tested by RGA established in the present disclosure and the pharmacopoeia method, 6 batches of samples are tested, and each sample is tested 3 times. The consistency of the method is evaluated by analyzing the relative biological activity values of the samples tested by each method. Analysis results are shown in FIG. 22. The relative biological activity of the two methods is statistically analyzed, P=0.4165 (P>0.05), indicating that RGA and the proliferation inhibition method assay have consistent determination results for evaluating the relative biological activity of rhIL-11. However, the mean of the relative biological activity of rhIL-11 measured by RGA is 98.33%, the CV is 4.41%, and all R² is greater than 0.99. The mean of the relative biological activity of rhIL-11 measured by the proliferation inhibition method in the pharmacopoeia is 94.51%, the CV is 94.51%, and all R² is greater than 0.97. The recovery rate and R² of RGA are greater than those of the proliferation inhibition method, indicating that the accuracy and reliability of the data of RGA are higher than those of the proliferation inhibition method, and RGA has the smaller CV, indicating that the precision and accuracy of RGA are higher. In conclusion, RGA is less time-consuming and more accurate than the proliferation inhibition method.

What is claimed is:

1. A method for rapidly testing biological activity of human interleukin-11 on the basis of luciferase reporter genes, comprising the following steps:
1. constructing cell strains based on luciferase reporter genes; and
2. preparing a sample to be tested into a gradient dilution solution, adding same into a culture solution, then, adding the culture solution into a culture medium containing the cell strains, and after co-incubation, testing the biological activity of human interleukin-11 by determining luciferase activity.

2. A construction method for cell strains based on luciferase reporter genes, comprising the following steps:
1. constructing pPR (EXP)-STAT3-luc2 plasmids, which comprises the specific steps:
1-1) synthesizing a promoter: obtaining STAT3 gene action element fragments;
1-2) performing connection and transformation: firstly, digesting a pPR(EXP)-luc2 vector with EcoR I and Sal I, then ligating the digested pPR(EXP)-luc2 vector with the STAT3 gene promoter fragment, and performing transformation into Escherichia coli competent cells after completion of ligation;
1-3) performing screening and verification: spreading the transformed Escherichia coli on a plate containing an antibiotic, performing inverted culture for 12-16 h, selecting bacteria for PCR identification, extracting plasmids from positive colonies, performing digestion to identify recombinant plasmids, sequence positive clones, and extract plasmids after sequencing and alignment is successful, thereby obtaining constructed recombinant expression plasmids of pPR(EXP)-STAT3-luc2;

2. transfecting cells with the plasmids of pPR(EXP)-STAT3-luc2, which comprises the specific steps:
2-1) performing recovery and culture of HEK293T cells: dissolving cryopreserved HEK293T cells in a 37° C. water bath, performing centrifugation for 5 min at 1360 rpm after the cells are dissolved, removing supernatant through suction, adding 1 mL of a complete medium of cells for resuspension, performing transfer into a T25 culture flask containing 5 mL of a complete medium of cells, and performing culture in an incubator;
2-2) performing cell transfection: seeding the HEK293T cells, performing culture for 24 h, then, adding the cells into a transfection system for culture for 48 h at 37° C., discarding a transfection solution, adding a culture solution, performing passaging at a density of 1:4 when the cells grow close to confluence, continuing culture, and performing positive monoclonal screening when cell confluency reaches 30% to obtain transfected cells;

3. screening stable cell lines, which comprises the specific steps:
3-1) screening puromycin: washing the transfected cells with phosphate buffer saline (PBS), performing digestion with trypsin, inoculating the cells into a culture dish, then, adding a culture medium containing puromycin, replacing the selective medium every 2-3 days, transferring the cells to a culture flask for continuous culture when the cell confluency is more than 90%, adding a culture medium containing puromycin, selecting monoclonal cells for continuous culture when the transfected cells grow to more than 70%, and adding a culture medium containing puromycin, wherein a concentration of the puromycin is reduced by half at this time; and
3-2) performing monoclonal selection and expanded culture: screening positive clones by a limited dilution method to obtain the cell strains.

3. The construction method for cell strains based on luciferase reporter genes according to claim 2, wherein in step 1-1), by consulting literature and NCBI, JASPAR is utilized to predict a promoter region, STAT3 related action elements are screened, four segments of sequences most closely related to human interleukin-11 action are selected, and cleavage sites of EcoRI and SalI are added at both ends of the sequences respectively after the sequences are connected in series as action elements.

4. The construction method for cell strains based on luciferase reporter genes according to claim 2, wherein in step 1-2), DH5α competent cells are used as the Escherichia coli competent cells.

5. The construction method for cell strains based on luciferase reporter genes according to claim 2, wherein in step 1-3), the antibiotic is ampicillin.

6. The construction method for cell strains based on luciferase reporter genes according to claim 2, wherein in step 2-1), the complete medium of cells is a Dulbecco's modified eagle medium (DMEM) culture solution containing double antibiotics of 10% fetal bovine serum and 1% streptomycin, and the culture conditions in the incubator are 5% CO₂ and 37° C.

7. The construction method for cell strains based on luciferase reporter genes according to claim 2, wherein in step 2-2), the transfection system comprises 2.5 μg of the pPR (EXP)-STAT3-luc2 plasmid, 1.25 μg of a transposon enzyme plasmid, 5 μL of a P3000 reagent, 3.75 μL of a Lipofectamine 3000 reagent and 250 μL of a DMEM.

8. The construction method for cell strains based on luciferase reporter genes according to claim 2, wherein in step 3-2), the steps of screening positive clones by the limiting dilution method comprise: preparing cell suspension, performing cell counting, diluting the cells to 5 cells/mL with a puro culture solution, adding the cell suspension into a 96-well plate at 200 μL/well, wherein a puro concentration is 25 μg/mL, then, adding the cell suspension at 100 μL/well, labeling monoclonal clones under a microscope, after cells in 96 wells are fully grown, transferring the cells into new wells for culture, performing repeated screening, and when the cultured cells basically do not die, performing sequencing identification to obtain the cell strains.

9. Application of the cell strains based on luciferase reporter genes according to claim 1 in testing of biological activity of human interleukin-11.


SEQUENCE LISTING (4 sequences)

SEQ ID NO: 1 - DNA, 18 bp, synthetic construct
  source 1..18: mol_type = other DNA
gtcgcattcccgtcaatc

SEQ ID NO: 2 - DNA, 22 bp, synthetic construct
  source 1..22: mol_type = other DNA
gctgtatttcctgaaagaagtt

SEQ ID NO: 3 - DNA, 17 bp, synthetic construct
  source 1..17: mol_type = other DNA
acgcgttacgggaatcg

SEQ ID NO: 4 - DNA, 21 bp, synthetic construct
  source 1..21: mol_type = other DNA
ggcaagttccaggaattaagc